(19) United States
(12) Patent Application Publication
(10) Pub. No.: US 2008/0153913 A1
(43) Pub. Date: Jun. 26, 2008

(54) Nutritional Supplement and Protocol

(75) Inventors: Ralph A. Cowden III, Zephyr Cove, NV (US); Alec Keith, Hilo, HI (US); James C. Roberts Jr., Toledo, OH (US); William E. Crisp, Paradise Valley, AZ (US); Janey A. Lau, Kaneohe, HI (US)

Correspondence Address: GODBEY GRIFFITHS REISS, LLLP, 1003 BISHOP STREET SUITE 2300, PAUAHI TOWER, HONOLULU, HI, 96813, US

(73) Assignee: Med Five, Inc., Honolulu, HI (US)

(21) Appl. No.: 10/571,740

(22) PCT Filed: Sep. 15, 2004
(86) PCT No.: PCT/US04/30056

§ 371 Date: Nov. 23, 2007

Related U.S. Application Data
(60) Provisional application No. 60/502,993, filed on Sep. 15, 2003.

(51) Int. Cl. A61K 31/685; A61K 38/46; A61K 31/195

(52) U.S. Cl. 514/566; 424/195.15; 514/440; 514/474; 514/78; 424/94.6

(57) ABSTRACT

Particular nutritional supplements and protocols emphasize the use of ethylenediamine tetraacetic acid as a mouth rinse, and as an enteric-coated oral pharmaceutical, further supplemented on a daily basis with both a combination of phosphatidyl lipids and alpha lipoic acid and a nutritional supplement containing approximately equal amounts of Reishi and Shiitake mushrooms.

### BACKGROUND OF THE INVENTION

1. Field of the Invention

The invention pertains to nutritional supplements that avert unwanted microbial action in the mouth, gastrointestinal tract and other patient tissues.

2. Description of Related Art

Naturopathy, orthodox pharmaceutical science, and even homeopathy are tending to converge and to agree, more and more, that wherever possible it is infinitely better to prevent disease than to treat it. While this idea seems to state the obvious, a decade or two or three before this was written western medicine was very much more focussed on disease treatments rather than prevention, and remnants of the disease-treatment prejudice survive to this day. The convergence in outlooks toward disease prevention is also represented by a consumer demand that wellness-oriented products be ubiquitous, effective, affordable and free from unwanted side effects. Seen in this context, the most important wellness products are those which address the issues of microbial culture and/or introduction into the oral cavity and consequent health ramifications of selective antimicrobial, toxin-abatement and other physiological processes.

Certain current pharmaceutical wellness initiatives are alarming to some practitioners of holistic healing. The appearance of Triclosan and other antibiotics in common toothpastes can be seen as yet another example of the overuse of antibiotics in United States health and grooming care, which overuse inevitably contributes to antibiotic resistance in the organism colonies sought to be controlled in the first place. Mercury and other preservatives in childhood and influenza vaccines are finally being acknowledged as potentially deleterious to health, with possible causal implications in autism, and elemental lead and other elemental toxins are a known environmental threat. Despite the health risks of elemental metal poisoning, little or no effort has been made in the traditional pharmaceutical industry to date to provide simple, home-use chelation therapy for mercury, lead and other toxins in a way which does not compromise the mineral-nutrient status of the patient being treated. It is therefore an object of this invention to provide a wellness oriented nutritional supplement and protocol which provides antimicrobial and antitoxin action as an adjunct to vitamin and mineral supplementation otherwise implemented by a patient.

### SUMMARY OF THE INVENTION

In order to meet this need, the present nutritional supplement and protocol embraces any one or more of four steps which can be regarded as “Clean Life,” “Defend Life,” “Renew Life” and “Protect Life.” The “Clean Life” stage is a mouth rinse which contains, per three pint amounts: 200-900 mg ethylene diamine tetraacetic acid (EDTA), preferably 445 mg; 5-50 mg ascorbic acid (Vitamin C), preferably 15 mg; 5-30 mg lysozyme, preferably 10 mg; and at least one excipient such as Croscarmellose Sodium and/or Magnesium Stearate. “Clean Life” product is generally formulated as a powder or solid, such as a tablet, which can be dissolved in the mouth rinse of the user's choice. “Defend Life” is a nutritional supplement which contains as active ingredients: EDTA 300-1600 mg daily, preferably 400 mg twice per day for a daily dose of 800 mg; 50-400 mg Ascorbic Acid, preferably 100 mg twice per day for a daily dose of 200 mg; and dimercaptosuccinic acid (DMSA) 10-30 mg daily, preferably 5 mg twice per day for a daily dose of 10 mg, all in enteric coated pharmaceutical dosage forms. “Defend Life” excipients may include Microcrystalline Cellulose, Silicon Dioxide, Magnesium Stearate and/or Di-Calcium Phosphate. “Renew Life” contains Phosphatidyl Lipid and Alpha Lipoic Acid, preferably in the form of gel caps for administration 4 times per day, two in the morning and two in the evening. “Protect Life” is a nutritional supplement containing both Reishi and Shiitake mushrooms in about equal parts, for administration in the range of about 150-1000 mg per day. In the most preferred embodiments of the invention, all of “Clean Life,” “Defend Life,” “Renew Life” and “Protect Life” are practiced by the patient every day with the exception that the enteric coated EDTA dosage forms are administered for five days with a two-day break each week. The invention embraces both the individual compositions and the treatment protocol with one or more of the four stages.

### DESCRIPTION OF THE PREFERRED EMBODIMENT(S)

The present nutritional supplement and protocol embraces any one or more of four steps which can be understood as “Clean Life,” “Defend Life,” “Renew Life” and “Protect Life.” Overall, the pharmaceutical dosage forms and/or methods of treating using one or more of the four dosage forms and treatment steps are designed to address oral hygiene, toxin and microbe removal, membrane repair and immune system stimulation. The “Clean Life,” “Defend Life,” “Renew Life” and “Protect Life” stages embrace both the underlying pharmaceutical dosage forms in their general and preferred embodiments and the protocols involved in administering one or more of the stages to a patient.

The “Clean Life” stage is a mouth rinse which contains, per three pints: 200-900 mg ethylene diamine tetraacetic acid (EDTA), preferably 445 mg; 5-50 mg Ascorbic Acid (Vitamin C), preferably 15 mg; 5-30 mg lysozyme, preferably 10 mg, and at least one excipient such as Croscarmellose Sodium and/or Magnesium Stearate. “Clean Life” product is generally formulated as a powder or solid, such as a tablet, which can be dissolved in the mouth rinse of the user's choice. Generally, a tablet is prepared such that 1 tablet can be placed in 1 pint of the mouth rinse. However, the amounts given here are for three pints of mouth rinse. After dissolving the above in the mouth rinse of choice, the user rinses with 1-3 teaspoons fall strength after brushing the teeth, with rinsing to occur for a fall thirty seconds. The mouth rinse should not be swallowed. It should be noted that in some commercial mouth rinses that contain greater than 9% alcohol (ethanol), the tablets might not dissolve completely. Ideally, a mouth rinse with less than 9% alcohol (ethanol) is selected for use with the invention. It should be understood that the EDTA content of the mouth rinse breaks down the plaque on the teeth. Lysozyme is an enzyme that attacks the carbohydrate chains forming protective cell walls of bacteria and which in turn causes the bacteria to burst under their own internal pressure. The presence of the Ascorbic Acid (Vitamin C) is as an antioxidant. Because most bacteria enter the body through the mouth, the antimicrobial nature of the mouth rinse described above promotes health and wellness. It is suggested that household partners both use the mouth rinse to prevent recurrence of nanobacteria or the exchange of bacteria between partners.

The “Clean Life” stage recognizes the importance of daily dental and oral hygiene in the prevention and management of heart disease and other diseases. Even though the administration of antibiotics to cardiac patients who have oral or gingival breaches is already routine at this writing, the invention takes a further prophylactic approach to reducing the entrance of bacteria into the body via the oral cavity in a daily prophylactic protocol which the inventors believe is as essential as daily brushing and flossing. The “Clean Life” stage works synergistically, it is believed, with the “Defend Life” stage discussed below.

“Defend Life” is a nutritional supplement which contains as active ingredients: EDTA 300-1600 mg daily, preferably 400 mg twice per day for a daily dose of 800 mg; 50-400 mg Ascorbic Acid (Vitamin C), preferably 100 mg twice per day for a daily dose of 200 mg; and dimercaptosuccinic acid (DMSA) 10-30 mg daily, preferably 5 mg twice per day for a daily dose of 10 mg, all in pharmaceutical dosage forms which are preferably enteric coated. “Defend Life” excipients may include Microcrystalline Cellulose, Silicon Dioxide, Magnesium Stearate and/or Di-Calcium Phosphate. “Defend Life” is to be practiced five days a week with a two-day break. Although any suitable pharmaceutical dosage form is feasible, generally tablets are prepared. Ideally, tablets contain 1000 mg of a combination of excipients which may include without limitation Microcrystalline Cellulose, Silicon Dioxide, Magnesium Stearate, Di-Calcium Phosphate, together with the preferred amounts of active ingredient per tablet, namely, 400 mg EDTA, 100 mg Ascorbic Acid (Vitamin C) and 5 mg DMSA, to be administered twice daily to an adult patient. In addition to the chelating effect of the EDTA, DMSA removes mercury from the body and eliminates it through the urine and feces. The literature also states that DMSA further helps to rid the body of harmful mycoplasms.

By way of explanation of the “Defend Life” portion of the instant protocol, it should be understood that the human physiological defense system is not designed to handle the amount of environmental toxins that it is routinely exposed to in air, water and food. Prior to the availability of the “Defend Life” protocol, it was not possible to address routine detoxification in the same way. The “Defend Life” active agents are preferably enteric coated and thus are not released in the acid environment of the stomach. Many existing detoxification agents at this writing are not enteric coated and frequently, as a result, cause stomach or gastrointestinal tract irritation and/or undergo alteration or destruction of the active component(s). By providing an enteric coated version of the composition used to implement “Defend Life,” it is believed that: 1) steady state delivery and constant blood levels are achieved; 2) excessive destruction of the active agents by the liver is avoided; and 3) regulation of blood levels of other active agents having low doses or short half-lives is optimized. The enteric coating dissolves almost immediately upon entry to the intestine, where the “Defend Life” actives are optimally absorbed. The synergy afforded between “Clean Life” and “Defend Life” forms the base line for additional synergy that occurs when preferably three stages, and most preferably all four stages, of the present protocol are implemented together. For example, Vitamin C taken alone serves as a beneficial antioxidant and helps to clear lead from the human system. Vitamin C in the present protocol does ameliorate lead but also enhances absorption of the “Defend Life” active agents and even (see below) the “Protect Life” active agents. Also, the Alpha Lipoic Acid (referred to below) has the joint action by itself providing metal binding effects (complementary to other metal chelating agents discussed herein) and by regenerating any Vitamin C that is altered due to physiologic processes and the anti-free-radical action of the Vitamin C itself.

“Renew Life” contains Phosphatidyl Lipid and Alpha Lipoic Acid, preferably in the form of gel caps for administration 4 times per day, two in the morning and two in the evening. The Phosphatidyl Lipid contains Phosphatidylethanolamine, Phosphatidylserine, Phosphatidylinositol and Phosphatidylcholine. The Phosphatidyl Lipid is administered in the amount of about 100-3500 mg per daily dose, preferably in the form of caplets containing 350 mg each and administered four times daily for a preferred daily dose of 1400 mg for an adult. The Alpha Lipoic Acid is administered in the amount of 3-300 mg per daily dose, preferably 25 mg in the same caplet as the Phosphatidyl Lipid for a preferred daily dose of 100 mg. The Phospholipids and Lipoic Acid mimic biological membrane structures that play a unique and critical role in biological processes. In the body, phospholipids are unequally distributed throughout the lipid and plasma membranes of nucleated cells. The Amino Phospholipids (Phosphatidylethanolamine (PE), Phosphatidylinositol (PI), and Phosphatidylserine (PS)) are located in the inner leaflet and Phosphatidylcholine (PC) are more abundant in the outer leaflet. PE, PI, PS and PC each have unique roles in the body and, as we age, these Phospholipids diminish and health suffers as a result. One of the Phosphatidyl Lipids, Phosphatidyl Choline (PC) will help to emulsify, to mobilize and to metabolize cholesterol, which causes cholesterol to become water soluble in the blood stream. Water soluble cholesterol is easily metabolized and/or eliminated from the body. PC also provides the body with necessary choline, deficiencies of which are well known etiologic factors in liver disease. Alpha Lipoic Acid is useful for cellular metabolism to combat many exposed chemicals and viruses, and is manufactured by the body (it is believed) as a crucial part of natural detoxification. Alpha Lipoic Acid controls different free radicals and restores various nutrients, namely, Vitamin E, Clutathione, Vitamin C, Enzyme CoQ10 (formerly known as Vitamin K) and various metabolic enzymes.

“Protect Life” is a nutritional supplement containing both Reishi and Shiitake mushrooms in about equal parts, for administration in the range of about 150-1000 mg per day. Reishi, or Ganoderma lucidum, is a traditional mushroom used to nourish, to tone and to supplement the whole body as it removes toxins. It also calms and nourishes the nervous system, strengthens the lungs, protects and stimulates the liver and helps the body to adapt to stress. Reishi helps to stimulate the immune system and is recognized as an adjunct for use as an immune system stimulator. The Reishi mushrooms contain high levels of polysaccharide that help to induce the production of interferon, which is a protein produced inside cells to fight viral infection. Reishi mushrooms also contain Adenosine, which helps to calm the central nervous system. Shiitake mushrooms, Lentinula edodes, is also recognized for its strong support of the immune system. The co-administration of Reishi and Shiitake is an important innovation of the present invention to achieve the desired nutritional and immunological result. Shiitake mushrooms are used in the far East to treat high blood pressure, to balance cholesterol levels, and as a kidney and liver tonic. Shiitake mushrooms are also believed to combat Herpes Simplex Type 1 virus, certain forms of cancer, environmental allergies, infections with Candida albicans and frequent colds and flu.

In the most preferred embodiments of the invention, all of “Clean Life,” “Defend Life,” “Renew Life” and “Protect Life” are practiced by the patient every day with the exception that the enteric coated EDTA dosage forms are administered for five days with a two-day break each week. The invention embraces both the individual compositions and the treatment protocol with one or more of the four stages.

The invention claimed is:

1. A nutritional supplement protocol in which effective amounts of ethylenediamine tetraacetic acid are administered both in a mouth rinse and as an oral pharmaceutical to a patient on at least every five out of seven days, and further wherein the ethylenediamine tetraacetic acid administered orally is enteric coated.

2. The nutritional supplement protocol according to claim 1, wherein the enteric coated ethylenediamine tetraacetic acid is administered, on the days that it is administered, in an amount between 200-900 mg.

3. The nutritional supplement protocol according to claim 1, wherein the mouth rinse containing ethylenediamine tetraacetic acid also contains Vitamin C.

4. The nutritional supplement protocol according to claim 1, wherein the mouth rinse containing ethylenediamine tetraacetic acid also contains Vitamin C and Lysozyme.

5. The nutritional supplement protocol according to claim 1, wherein an additional dose of phosphatidyl lipids is administered to the patient on a daily basis.

6. The nutritional supplement protocol according to claim 5, wherein said additional dose of phosphatidyl lipids is in the daily range of about 100-3500 mg for an adult patient.

7. The nutritional supplement protocol according to claim 6, wherein said additional dose of phosphatidyl lipids is in the daily range of about 1400 mg.

8. The nutritional supplement protocol according to claim 7, wherein said additional dose of phosphatidyl lipids is accompanied by a 3-300 mg dose of alpha lipoic acid.

9. The nutritional supplement protocol according to claim 8, wherein a further daily dose of a combination of Reishi and Shiitake mushrooms is administered to the patient.

10. A kit for nutritional supplementation containing any or all of: a) a soluble ethylenediamine tetraacetic acid pharmaceutical dosage form suitable for solution or suspension in a mouth rinse; b) at least one oral, enteric-coated pharmaceutical dosage form containing ethylenediamine tetraacetic acid; at least one oral dosage form containing phosphatidyl lipids; and at least one oral dosage form containing a combination of Reishi and Shiitake mushrooms in approximately equal proportions.
